(19) United States
(12) Patent Application Publication
(10) Pub. No.: US 2006/0121094 A1
(43) Pub. Date: Jun. 8, 2006

(54) Preparation comprising 1,3 and/or 1,6 beta glucans for the treatment of infections and inflammations in animals

(76) Inventor: Mark Geach, London (GB)

Correspondence Address: NIXON & VANDERHYE, PC, 901 NORTH GLEBE ROAD, 11TH FLOOR, ARLINGTON, VA, 22203, US

(21) Appl. No.: 10/537,047

(22) PCT Filed: Nov. 3, 2003
(86) PCT No.: PCT/GB03/04735

§ 371 Date: Feb. 8, 2006

(30) Foreign Application Priority Data
Nov. 1, 2002 (GB) 0225502.4

(51) Int. Cl. A61K 31/715; A61K 31/665; A61K 36/06; A61K 36/02

(52) U.S. Cl. 424/442; 514/54; 514/100; 424/195.16; 424/195.17

(57) ABSTRACT

The present invention provides a preparation comprising 1,3 and/or 1,6 beta glucans, for use in the treatment or prevention of infection, inflammation, and/or traumatic conditions. The preparation may be used in bath or immersion treatment, or can be administered orally or by injection.

The present invention relates to preparations for the treatment or prevention of clinical conditions in animals and, in particular, in aquatic animals.

The management of clinical conditions for animals, particularly the management of clinical conditions in fish, invertebrates and amphibians is very important, both in terms of animal well-being and in commercial terms where the animals are sold or displayed and clearly need to be in good health.

However, the treatment or prophylaxis of clinical conditions such as inflammation in animals can often be problematic and it is desirable to keep the disturbance to the animals to a minimum.

According to the present invention, a preparation comprising 1,3 and/or 1,6 beta glucans is provided, for use in the treatment or prevention of infection, inflammation, and/or traumatic conditions.

1,3 and/or 1,6 beta glucans are commercially available under the name MacroGard® from Biotec ASA (Tromsø, Norway). The 1,3 and/or 1,6 beta glucans are derived from Baker's yeast (Saccharomyces cerevisiae). Beta glucans may, of course, be derived from other sources.

In a preferred embodiment, the 1,3 and/or 1,6 beta glucans in the preparation are water soluble.

In another preferred embodiment, the preparation further comprises vitamins, minerals, carotenoids, amino acids, lipids, peptides, nucleotides, bioflavinoids, oils and/or polysaccharides. The inclusion of vitamins such as vitamin C, particularly as ascorbyl polyphosphate, is especially effective where the formulation is used to treat fish. The beta glucans appear to potentiate the effects of these further components, making the preparations particularly effective in wound healing and the like.

In yet another preferred embodiment, the preparation comprises water insoluble 1,3 and/or 1,6 beta glucans, in addition to or instead of the water soluble beta glucans. The water insoluble 1,3 and/or 1,6 beta glucans are preferably in microparticulate form.

Other preferred preparations further comprise microparticulate yeast and/or algae. The preferred algae to be used in the preparations of the present invention include Aphanizomenon flosaquae. The yeast used may be of the Phaffia species and a source of astaxanthin.

In yet another preferred embodiment of the present invention, the preparation further comprises additives having bacteriostatic or bacteriocidal properties, such as formaldehyde.

It has previously been disclosed that 1,3 and/or 1,6 beta glucans have immunostimulatory properties when administered to fish. It has now been discovered that 1,3 and/or 1,6 beta glucans are highly beneficial in the management of infective, traumatic and inflammatory conditions in animals such as fish and invertebrates. They are also useful in the management of skin and fin conditions in fish. It has further been discovered that the 1,3 and/or 1,6 beta glucans improve food utilisation and so are particularly useful in farming and breeding of ornamental fish.

The preparations of the present invention have also been shown to potentiate the immunostimulatory and/or anti-inflammatory properties of other treatments administered to the animals.

The preparation according to the invention may be used in bath or immersion treatment, or can be administered orally or by injection.

Bath, immersion or direct topical treatment has been found to be effective in the management of clinical conditions for animals, particularly for the management of clinical conditions in fish, invertebrates and amphibians. The preparation according to the present invention may be administered as a short term bath or immersion treatment, or may be added to water systems containing animals for longer term applications.

The beta glucans used in a preparation for bath or immersion treatment are preferably soluble. Soluble 1,3 and/or 1,6 beta glucans are commercially available under the name MacroGard® AquaSol from Biotec ASA (Tromsø, Norway). The 1,3 and/or 1,6 glucans are derived from Baker's yeast (Saccharomyces cerevisiae) and are processed to render them soluble.

The soluble 1,3 and/or 1,6 beta glucans are added to the water, for example in an amount of 1 ml per litre of water, for treatment of fish over a period of 1-24 hours. When used in this way, the preparation according to the present invention has been shown to produce a marked increase in the rate of wound healing and reduces associated inflammation. It has also been shown to trigger or increase immunostimulation, which is observed in particular in the skin or mucus coating.

Use of the preparation according to the present invention as a bath additive has also been shown to have a beneficial effect, improving the mucus quality in fish. It has also been note that the use of preparations according to the present invention frequently leads to a reduction in the bacterial and pathogen count in scrapings from the fishes' mucus coating. What is more, these beneficial effects and improvements are observed in the entire fish population and not just in a small number of individuals within the population.

Bath or immersion treatment clearly has the advantage that a number of animals may be treated together rather than requiring individual attention. What is more, the animals may not even be aware that they are being treated. The preparation according to the present invention has been used as a bath treatment for a very wide range of marine and fresh water fish and has been found to have excellent safety during and following application.

When the preparation of the present invention is to be administered by injection, this may be by subcutaneous, intramuscular or intravenous injection.

Pure 1,3 and/or 1,6 beta glucans have been administered by injection in an amount of 0.01-0.2 mls per 100 g bodyweight for the management of inflammatory and/or infective conditions in fish. Such administration has shown a marked increase in the rate of wound resolution, reducing the effects of toxins and appearing to have a marked anti-inflammatory effect. The effects of treatment have been very noticeable in cases of bacterial infections, particularly with Vibrio species and most noticeably with Vibrio vulnificus (which is known to produce exotoxins).

When the preparation according to the invention is intended for administration by injection, it is preferable to use soluble 1,3 and/or 1,6 beta glucans.

The preparation according to the present invention may also be administered orally. The 1,3 and/or 1,6 beta glucans may be either in the soluble or non-soluble form. The preparation may be formulated into liquid, gel or solid form. Particularly preferable are tablets, (hard or soft shell) capsules, liquids, gels and powders. The preparation may be added to the animal feed, for example by injection or pressure treatment of feed, or simply by sprinkling the powder or liquid form onto feed.

The preparations can be prepared as gels using conventional techniques. Preferred gelling agents which can be used include carrageenans and carboxymethylcellulose, and the like.

Topical administration of the preparation according to the present invention has also been observed to have good wound healing effects and may be used on various types of animals.

In order to increase the shelf life of the formulation according to the present invention, the formulation may include conventional preservatives.

In a preferred embodiment, the preparation according to the present invention includes an excipient and preferably a pharmaceutically acceptable excipient.

The formulations according to the present invention have been found to be particularly useful in preventing deterioration of the clinical condition of fish, including ornamental and farmed fish, as a result of their transportation. The preparation is simply administered in one of the ways described above prior to transportation of the fish. The preparations may additionally or alternatively be administered after transportation. A particularly beneficial use of the preparations according to the present invention is a manner of quarantine treatment, whereby the fish are immersion treated for 1-24 hours immediately prior to being introduced into a new environment. This type of treatment has been observed to protect the fish when they are introduced into their new environment.

1. A preparation comprising 1,3 and/or 1,6 beta glucans, for use in the treatment or prevention of infection, inflammation, and/or traumatic conditions in an animal.

2. A preparation as claimed in claim 1, wherein at least some of the 1,3 and/or 1,6 beta glucans are water soluble.

3. A preparation as claimed in claim 1, wherein at least some of the 1,3 and/or 1,6 beta glucans are not water soluble.

4. A preparation as claimed in claim 3, wherein the 1,3 and/or 1,6 beta glucans which are not water soluble are in microparticulate form.

5. A preparation as claimed in claim 1, wherein the preparation further comprises vitamins, minerals, carotenoids, amino acids, lipids, peptides, nucleotides, bioflavinoids, oils, yeasts, algae and/or polysaccharides.

6. A preparation as claimed in claim 5, wherein the preparation further comprises vitamin C, preferably as ascorbyl polyphosphate.

7. A preparation as claimed in claim 1, further comprising an additive having bacteriostatic or bacteriocidal activity.

8. A preparation as claimed in claim 1, wherein the preparation is for administration by injection.

9. A preparation as claimed in claim 1, wherein the preparation is for use in bath or water immersion treatment.

10. A preparation as claimed in claim 1, wherein the preparation is for oral administration.

11. A preparation as claimed in claim 10, wherein the preparation is in the form of a powder, gel, liquid, tablet or capsule.

12. A preparation as claimed in claim 10, wherein the preparation is added to animal feed.

13. A preparation as claimed in claim 1, wherein the preparation is for topical administration.

14. A preparation as claimed in claim 1, wherein the preparation is substantially soluble in water.

15. A preparation as claimed in claim 1, wherein the animal is an aquatic animal.
